(19) United States
(12) Patent Application Publication
(10) Pub. No.: US 2021/0321203 A1
(43) Pub. Date: Oct. 14, 2021

(54) HEARING COMPENSATION MODULE

(71) Applicant: Scent Blaster Limited, Hong Kong (CN)

(72) Inventor: Liyan YIN, Ankang City (CN)

(21) Appl. No.: 17/167,094

(22) Filed: Feb. 4, 2021

(30) Foreign Application Priority Data
Apr. 9, 2020 (CN) 202020508764.2

(51) Int. Cl. H04R 25/00; A61B 5/12; G16H 10/60

(52) U.S. Cl. CPC H04R 25/50; H04R 2225/55; G16H 10/60; A61B 5/12

(57) ABSTRACT

A hearing compensation module disposed in a voice service module includes a control unit, a voice receiving unit, a voice playback unit, a storage unit, and an identity recognition module. The storage unit stores a hearing compensation datum corresponding to a user. The identity recognition module adjusts an access authority of the control unit to the hearing compensation datum. The voice receiving unit receives a first voice message through the user. The control unit converts the first voice message into a second voice message according to the hearing compensation datum. The voice playback unit plays the second voice message to the user.

### BACKGROUND

### Technical Field

The present disclosure relates to a hearing compensation module, and more particularly to a hearing compensation module that can adjust voice services in real time according to hearing health states of different users.

### Description of Related Art

The statements in this section merely provide background information related to the present disclosure and do not necessarily constitute prior art.

For a long time, as the problems of aging societies in various countries have gradually emerged, various degenerative problems or diseases have gradually been paying attention. However, among various health promotion issues, hearing degradation is usually ignored. According to the world health organization (WHO), one third of elderly people over 65 years old has a hearing impairment. As many people grow older, their hearing will also deteriorate. Due to differences in physique, some people still have good ears and eyes when they are 90 years old, and some people lose their hearing after they are 50 years old. External factors that affect hearing include trauma to the skull, environmental noise, cardiovascular disease, diabetes, drugs, or viral infections of the inner ear, etc., are also likely to cause hearing impairment. Therefore, many elderly people often have psychological problems after hearing impairment, such as alienating relatives and friends, refusing to socialize, withdrawn and suspicious, depression, delusion, irritability, etc.

In particular, voice devices are now commonly used in smart homes and digital electronic products that connect various functions. Since voice service modules (such as voice assistants, voice services of telephone, or other related voice service devices, etc.) that can respond to people are not adjusted for people with poor or impaired hearing, it is easy to cause response troubles and various inconveniences in life for people with poor or impaired hearing to use ordinary voice service modules.

Therefore, how to design a hearing compensation module that can perform different hearing compensation adjustments for different users when using voice service modules, and more particularly to solve the aforementioned conventional technical problems of ordinary voice service modules are not adjusted for people with poor or impaired hearing, and being easy to cause response troubles and various inconveniences in life for people with poor or impaired hearing, etc., was studied by inventor of the present disclosure.

### SUMMARY

A purpose of the present disclosure is to provide a hearing compensation module that solves the technical problems of ordinary voice service modules are not adjusted for people with poor or impaired hearing, and being easy to cause response troubles and various inconveniences in life for people with poor or impaired hearing, etc. in the prior art, so as to achieve the purpose of allowing people with poor or impaired hearing to easily respond to voice service modules.

In order to achieve the purpose, the hearing compensation module disposed in a voice service module, the hearing compensation module includes a control unit, a voice receiving unit, a voice playback unit, a storage unit, and an identity recognition module. The control unit converts a first voice message into a second voice message according to a hearing compensation datum. The voice receiving unit is coupled to the control unit, the voice receiving unit receives the first voice message through a user, and transmits the first voice message to the control unit. The voice playback unit is coupled to the control unit, the voice playback unit receives the second voice message through the control unit, and plays the second voice message to the user. The storage unit is coupled to the control unit, and stores the hearing compensation datum corresponding to the user. The identity recognition module is coupled to the control unit, the identity recognition module receives an identity recognition datum from the user, and adjusts an access authority of the control unit to the hearing compensation datum according to the identity recognition datum.

Further, in the hearing compensation module, the identity recognition datum includes e-mail information, password information, fingerprint information, palmar digital vein information, voiceprint information, face image information, or retina information.

Further, in the hearing compensation module, the identity recognition module includes a server, a workstation, a fingerprint sensor, a finger vein sensor, a microphone, a camera, or a retina sensor.

Further, in the hearing compensation module, the voice receiving unit receives the first voice message generated by a human vocal cord, an artificial larynx, or an assistive context-aware toolkit (ACAT).

Further, in the hearing compensation module, further includes an assistive context-aware toolkit (ACAT), the ACAT is coupled to the control unit, and the voice receiving unit receives the first voice message from the user through the ACAT.

Further, in the hearing compensation module, the voice playback unit includes a speaker, an in-ear headphone, a headphone, an air conduction hearing aid, a bone conduction hearing aid, or a middle ear implant.

Further, in the hearing compensation module, further includes a display unit, the display unit is coupled to the control unit, and displays the identity recognition datum or the hearing compensation datum.

Further, in the hearing compensation module, further includes a power unit, the power unit is coupled to the control unit.

Further, in the hearing compensation module, when the user inputs the identity recognition datum to the identity recognition module, the storage unit receives the hearing compensation datum of the user.

Further, in the hearing compensation module, the hearing compensation datum includes a lowest audible sound pressure level of at least one of the user's left ear and right ear when the frequencies are 500 Hz, 1000 Hz, 2000 Hz, and 4000 Hz, respectively.

When using the hearing compensation module of the present disclosure, the voice receiving unit disposed in the voice service module receives the first voice message by user, and the voice receiving unit transmits the first voice message to the control unit. At the same time, before or after, the identity recognition module receives the identity recognition datum from the user, and adjusts the access authority of the control unit to the hearing compensation datum according to the identity recognition datum. The hearing compensation datum may include a hearing curve for a spectral range. Afterward, if the identity recognition module determines that current user is a correct user based on the identity recognition datum, and confirms that the storage unit has stored the hearing compensation datum that matches the current user, the control unit converts the first voice message into the second voice message according to the hearing compensation datum. The conversion may be to reversely adjust frequency spectrum of the first voice message according to the hearing curve in the hearing compensation datum to compensate for poor part of the current user's hearing. Finally, the voice playback unit plays the second voice message to the current user, so that the current user can hear a voice message that has been compensated for the poor part of the current user's hearing.

For this reason, the present disclosure solves the prior art technical problems about ordinary voice service module is not adjusted for people with poor or impaired hearing, and being easy to cause response troubles and various inconveniences with the voice service module in life for people with poor or impaired hearing, etc. in the prior art, so as to achieve the purpose of allowing people with poor or impaired hearing to easily respond to the voice service module.

In order to further understand the techniques, means, and effects of the present disclosure for achieving the intended purpose. Please refer to the following detailed description and drawings of the present disclosure. The drawings are provided for reference and description only, and are not intended to limit the present disclosure.

### BRIEF DESCRIPTION OF DRAWINGS

FIG. 1 and FIG. 2 are schematic diagrams of a first embodiment of a hearing compensation module of the present disclosure.

FIG. 3 is a schematic structural diagram of a second embodiment of the hearing compensation module of the present disclosure.

FIG. 4 is a schematic structural diagram of a third embodiment of the hearing compensation module of the present disclosure.

FIG. 5 is a schematic diagram of a hearing compensation datum of the hearing compensation module of the present disclosure.

### DETAILED DESCRIPTION

The technical content and detailed description of the present disclosure will be described below in conjunction with the drawings.

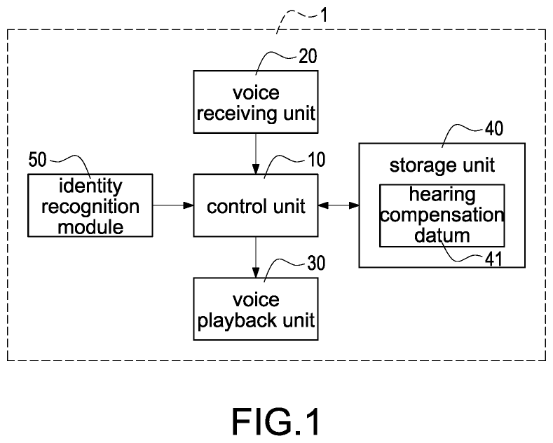

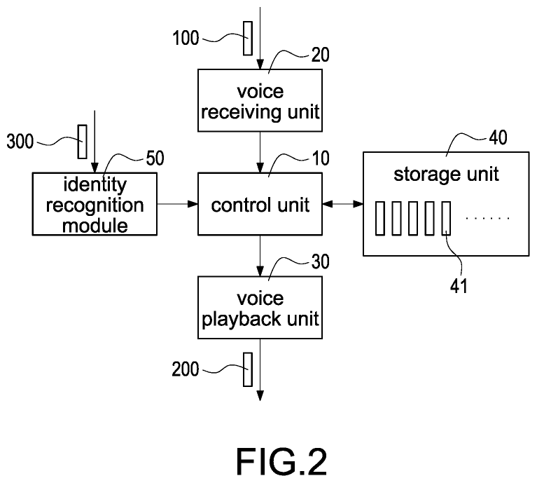

Please refer to FIG. 1 and FIG. 2, which are schematic diagrams of a first embodiment of a hearing compensation module of the present disclosure. The hearing compensation module is disposed in a voice service module 1, and the hearing compensation module includes a control unit 10, a voice receiving unit 20, a voice playback unit 30, a storage unit 40, and an identity recognition module 50.

The control unit 10 converts a first voice message 100 into a second voice message 200 according to a hearing compensation datum 41. The control unit 10 may include one of a microcontroller (MCU), a microprocessor (MPU), a central processing unit (CPU), an application-specific integrated circuit (ASIC), and a system-on-chip (SoC). The microcontroller may also include a circuit board based on the Arduino machine code architecture. The system-on-chip may be also a Raspberry Pi, and its model can be 1A, 1A+, 1B, 1B+, 2B, 3B, 3B+, 3A+, or 4B. The conversion may be to reversely adjust frequency spectrum of the first voice message 100 according to a hearing curve in the hearing compensation datum 41 to compensate for poor part of the current user's hearing.

The voice receiving unit 20 is coupled to the control unit 10, receives the first voice message 100 through a user (no shown), and transmits the first voice message 100 to the control unit 10. The voice receiving unit 20 may be a general dynamic microphone, a condenser microphone, an electret condenser microphone, a microelectromechanical microphone (mems microphone), an aluminum ribbon microphone, or carbon microphone. Moreover, the voice receiving unit 20 operates in modes of an omnidirectional, cardioid/hyper cardioid, or bi-directional/figure-of-8. The voice receiving unit 20 receives the first voice message 100 generated by a human vocal cord, an artificial larynx, or an assistive context-aware toolkit (ACAT). The first voice message 100 may be Mandarin, Minnan dialect, Hakka, or dialects such as Cantonese, Northeastern dialect, Henan dialect, Shaanxi dialect, Tianjin dialect, etc. However, the present disclosure is not limited thereof.

The voice playback unit 30 is coupled to the control unit 10, receives the second voice message 200 through the control unit 10, and plays the second voice message 200 to the user. The voice playback unit 30 includes a speaker, an in-ear headphone, a headphone, an air conduction hearing aid, a bone conduction hearing aid, or a middle ear implant. The speakers may be also electrodynamic speakers, electromagnetic speakers, piezoelectric speakers, electrode speakers, or plasma arc speakers. However, the present disclosure is not limited thereof.

The storage unit 40 is coupled to the control unit 10, and stores the hearing compensation datum 41 corresponding to the user. In the first embodiment of the present disclosure, the number of the hearing compensation datum 41 is plural to correspond to multiple different users. The storage unit 40 is an EEPROM, a NAND Flash, or other non-volatile data storage medium. Further, the hearing compensation datum 41 may include a lowest audible sound pressure level (SPL) of at least one of the left ear and the right ear of the user at frequencies of 500 Hz, 1000 Hz, 2000 Hz, and 4000 Hz, respectively, and the unit of the lowest audible SPL is dB (SPL). In the first embodiment of the present disclosure, and the storage unit 40 is a local storage device, a cloud server, a cloud workstation, or a cloud database, etc.

The identity recognition module 50 is coupled to the control unit 10, receives an identity recognition datum 300 from the user, and adjusts an access authority of the control unit 10 to the hearing compensation datum 41 according to the identity recognition datum 300. In the first embodiment of the present disclosure, the identity recognition datum 300 may include e-mail information, password information, fingerprint information, palmar digital vein information, voiceprint information, face image information, or retina information. The identity recognition module 50 may include a server, a workstation, a fingerprint sensor, a finger vein sensor, a microphone, a camera, or a retina sensor.

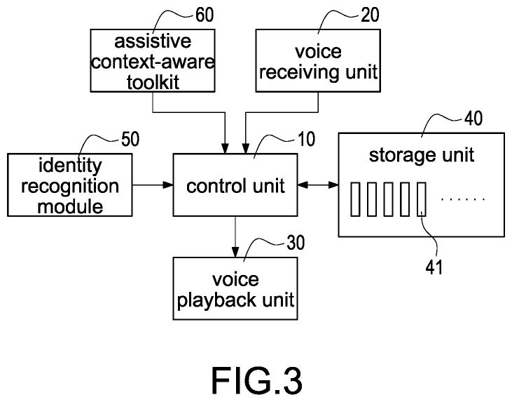

Please refer to FIG. 3, which is a schematic structural diagram of a second embodiment of the hearing compensation module of the present disclosure. The second embodiment of the present disclosure is substantially the same as the first embodiment of the present disclosure, except that the hearing compensation module more includes the ACAT 60. The ACAT 60 is coupled to the control unit 10, and the voice receiving unit 20 receives the first voice message 100 from the user through the ACAT 60.

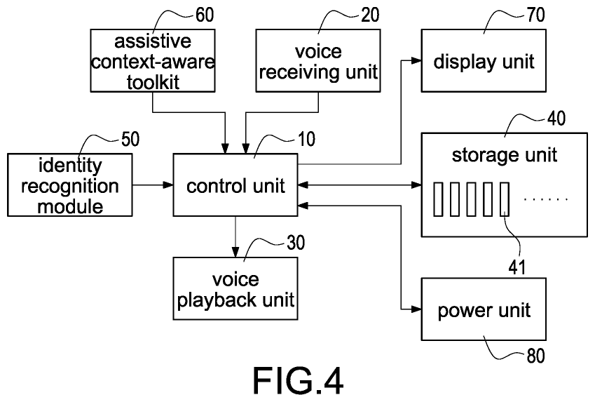

Please refer to FIG. 4, which is a schematic structural diagram of a third embodiment of the hearing compensation module of the present disclosure. The third embodiment of the present disclosure is substantially the same as the second embodiment of the present disclosure, except that the hearing compensation module more includes a display unit 70 and a power unit 80. The display unit 70 is coupled to the control unit 10, and displays the identity recognition datum 300 or the hearing compensation datum 41. The power unit 80 is coupled to the control unit 10, and is used to supply electric energy to the control unit 10. The display unit 70 may be composed of a liquid crystal display (LCD), a light emitting diode (LED), an organic light emitting diode (OLED), a quantum dot organic light emitting diode (QD-OLED), a mini-LED, a micro-LED, an indium gallium zinc oxide (IGZO), or an electronic paper (e-Paper). The power unit 80 may be a lithium battery, a nickel-hydrogen battery, a hydrogen-oxygen battery, a lead-acid battery, etc.

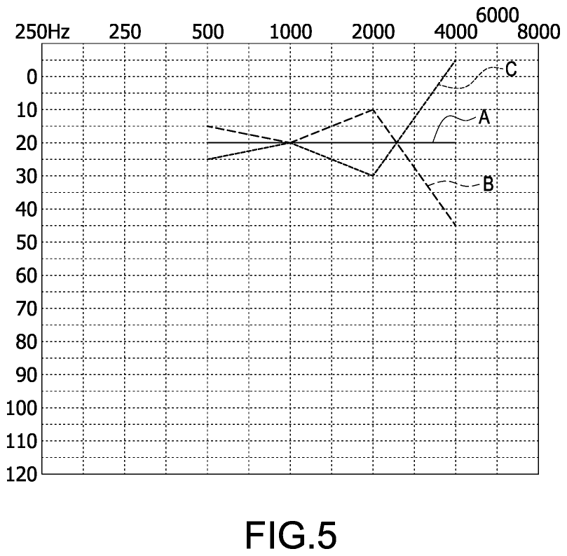

Please refer to FIG. 5, which is a schematic diagram of a hearing compensation datum 41 of the hearing compensation module of the present disclosure. In any embodiment of the present disclosure, the hearing compensation datum 41 may mainly include the lowest audible SPL that may be audible at 500 Hz, 1000 Hz, 2000 Hz, and 4000 Hz. As shown in FIG. 5, a hearing curve of a normal person in an ideal state should be A. The lowest audible SPL of the hearing curve A at 500 Hz, 1000 Hz, 2000 Hz, and 4000 Hz is 20 dB (SPL). It is assumed that the hearing compensation datum 41 measured by the current user appears is a hearing curve B. The lowest audible SPL of the hearing curve B at 500 Hz, 1000 Hz, 2000 Hz, and 4000 Hz are 15 dB (SPL), 20 dB (SPL), 10 dB (SPL), 45 dB (SPL), respectively. At this point, it can be clearly understood that the current user's hearing is poor in the high frequency region, especially at 4000 Hz. The control unit 10 shown in FIG. 1 reversely adjusts frequency spectrum of the first voice message 100 according to the hearing curve in the hearing compensation datum 41. For current users, it is mainly based on the hearing curve B to increase the SPL of 4000 Hz to higher than 500 Hz, 1000 Hz, and 2000 Hz. That is, a hearing curve C is used to compensate the weak part in 4000 Hz of the current user's hearing, so that the current user feels that 500 Hz, 1000 Hz, 2000 Hz and 4000 Hz are all the same loud when listening to the second voice message 200, and the current user has same sensitivity to each frequency band.

When the user uses the hearing compensation module of the present disclosure, the voice receiving unit 20 disposed in the voice service module 1 receives the first voice message 100 by the user, and the voice receiving unit 20 transmits the first voice message 100 to the control unit 10. At the same time, before or after, the identity recognition module 50 receives the identity recognition datum 300 from the user, and adjusts the access authority of the control unit 10 to the hearing compensation datum 41 according to the identity recognition datum 300. The hearing compensation datum 41 may include a hearing curve for a spectral range. Afterward, if the identity recognition module 50 determines that the current user is a correct user based on the identity recognition datum 300, and confirms that the storage unit 40 stored the hearing compensation datum 41 that matches the current user, the control unit 10 converts the first voice message 100 into the second voice message 200 according to the hearing compensation datum 41. The conversion may be to reversely adjust frequency spectrum of the first voice message 100 according to the hearing curve in the hearing compensation datum 41 to compensate for poor part of the current user's hearing. Finally, the voice playback unit 30 plays the second voice message 200 to the current user, so that the current user can hear a voice message that has been compensated for the poor part of the current user's hearing.

For this reason, the present disclosure can solve the prior art technical problems about ordinary voice service module are not adjusted for people with poor or impaired hearing, and being easy to cause response troubles and various inconveniences with the voice service module 1 in life for people with poor or impaired hearing, etc. in the prior art, so as to achieve the purpose of allowing people with poor or impaired hearing to easily respond to the voice service module 1.

The above is only a detailed description and drawings of the preferred embodiments of the present disclosure, but the features of the present disclosure are not limited thereto, and are not intended to limit the present disclosure. All the scope of the present disclosure shall be subject to the scope of the following claims. The embodiments of the spirit of the present disclosure and its similar variations are intended to be included in the scope of the present disclosure. Any variation or modification that can be easily conceived by those skilled in the art in the field of the present disclosure can be covered by the following claims.

It should be understood that the structures, the proportions, the sizes, the number of components, and the like in the drawings are only used to cope with the contents disclosed in the specification for understanding and reading by those skilled in the art, and it is not intended to limit the conditions that can be implemented in the present disclosure, and thus is not technically significant. Any modification of the structure, the change of the proportional relationship, or the adjustment of the size, should be within the scope of the technical contents disclosed by the present disclosure without affecting the effects and the achievable effects of the present disclosure.

What is claimed is:

1. A hearing compensation module disposed in a voice service module, the hearing compensation module comprising:
a control unit configured to convert a first voice message into a second voice message according to a hearing compensation datum,
a voice receiving unit coupled to the control unit, the voice receiving unit configured to receive the first voice message through a user, and transmit the first voice message to the control unit,
a voice playback unit coupled to the control unit, the voice playback unit configured to receive the second voice message through the control unit, and play the second voice message to the user,
a storage unit coupled to the control unit, and configured to store the hearing compensation datum corresponding to the user, and
an identity recognition module coupled to the control unit, the identity recognition module configured to receive an identity recognition datum from the user, and adjust an access authority of the control unit to the hearing compensation datum according to the identity recognition datum.

2. The hearing compensation module as claimed in claim 1, wherein the identity recognition datum comprises e-mail information, password information, fingerprint information, palmar digital vein information, voiceprint information, face image information, or retina information.

3. The hearing compensation module as claimed in claim 1, wherein the identity recognition module comprises a server, a workstation, a fingerprint sensor, a finger vein sensor, a microphone, a camera, or a retina sensor.

4. The hearing compensation module as claimed in claim 1, wherein the voice receiving unit is configured to receive the first voice message generated by a human vocal cord, an artificial larynx, or an assistive context-aware toolkit.

5. The hearing compensation module as claimed in claim 1, further comprising:
an assistive context-aware toolkit coupled to the control unit, and the voice receiving unit configured to receive the first voice message from the user through the assistive context-aware toolkit.

6. The hearing compensation module as claimed in claim 1, wherein the voice playback unit comprises a speaker, an in-ear headphone, a headphone, an air conduction hearing aid, a bone conduction hearing aid, or a middle ear implant.

7. The hearing compensation module as claimed in claim 1, further comprising:
a display unit coupled to the control unit, and the display unit configured to display the identity recognition datum or the hearing compensation datum.

8. The hearing compensation module as claimed in claim 1, further comprising:
a power unit coupled to the control unit.

9. The hearing compensation module as claimed in claim 1, wherein when the user inputs the identity recognition datum to the identity recognition module, the storage unit is configured to receive the hearing compensation datum of the user.

10. The hearing compensation module as claimed in claim 1, wherein the hearing compensation datum comprises a lowest audible sound pressure level of at least one of the user's left ear and right ear when the frequencies are 500 Hz, 1000 Hz, 2000 Hz, and 4000 Hz, respectively.
